(19) United States
(12) Patent Application Publication
(10) Pub. No.: US 2023/0105385 A1
(43) Pub. Date: Apr. 6, 2023

(54) Method And Assembly For Predicting The Occurrence Of Heart Disease Within An Individual

(71) Applicants: Subramaniam Ganesan, Rochester Hills, MI (US); Saad M. Almutairi, Tabuk City (SA); Manimurugan Shanmuganathan Eswari, Tabuk City (SA); Majed Mohammed Aborokbah, Tabuk City (SA); Narmatha Chellamani Maheswari, Tabuk City (SA); Riyadh A. Alzahebhani, Tabuk City (SA); Hani Sayyaf Almoamari, Tabuk City (SA)

(72) Inventors: Subramaniam Ganesan, Rochester Hills, MI (US); Saad M. Almutairi, Tabuk City (SA); Manimurugan Shanmuganathan Eswari, Tabuk City (SA); Majed Mohammed Aborokbah, Tabuk City (SA); Narmatha Chellamani Maheswari, Tabuk City (SA); Riyadh A. Alzahebhani, Tabuk City (SA); Hani Sayyaf Almoamari, Tabuk City (SA)

(21) Appl. No.: 17/300,719

(22) Filed: Oct. 1, 2021

(51) Int. Cl. A61B 5/00; A61B 5/021; A61B 5/024

(52) U.S. Cl. CPC A61B 5/7275; A61B 5/021; A61B 5/024; A61B 5/6801; A61B 5/7285

(57) ABSTRACT

An assembly 10 and a method for predicting the occurrence of heart disease within an individual 12 by the combined use of echocardiogram data and bodily metric data, such as that which may be obtained from a wearable device 18.

### GENERAL BACKGROUND

### Field of the Invention

The present inventions generally relate to a prediction method and a prediction assembly and more particularly, to a method and an assembly which predicts the occurrence of heart disease within an individual.

### Background of the Invention

Heart disease is a very serious and often fatal condition which affects millions of individuals annually. Oftentimes, individuals have a heart disease condition without realizing it and this undetected condition oftentimes worsens and suddenly causes, what is referred to as a “heart attack”, which is often either fatal or leaves the heart muscle so damaged as to place the individual into a “heart failure” mode which greatly and detrimentally effects the overall quality of that individual's life. As used herein, the term “heart disease” refers to plaque build-up or another type of blockage within one or more of the arteries and/or vessels “feeding” the heart or communicating with the heart.

If it is known that an individual has an occurrence of heart disease before a heart attack occurs, then that individual could be given statins (or another drug regimen), and inter-alia be placed on a low fat and low cholesterol diet to greatly reduce the probability of a heart attack occurring. Such pro-active intervention may also include the placement of a stent or similar device within one or more blocked arteries or vessels and the initiation of a regular aerobic exercise regime. All of the foregoing interventions may even reverse the heart disease in addition to reducing its progression. Such “pro-active” intervention would save lives. However, this “pro-active” intervention depends upon the ability to predict or discover that an individual actually has heart disease before the heart disease causes damage.

One way of discovering the occurrence of heart disease within an individual is to perform an invasive catherization on that individual during which a catheter/camera assembly is inserted into the body of the individual and threaded through that individual's arteries and/or vessels. While this invasive approach is accurate, it is costly and has the potential to harm the individual. Further, this invasive approach must be repeated over time in order to determine whether heart disease is present or has progressed and it is known that the probability of a serious side effect occurs as the number of these procedures are accomplished on an individual. For the foregoing reasons, catherizing the general public has never been adopted or considered a viable approach to reducing the devastating impact that heart disease has on the general public.

Other non-invasive approaches to determining the existence of heart disease exist, such a radiographic whole-body scan exist, but they require exposing the individual to radiation, injecting dye into the individual which could damage the kidneys of that individual, are known to miss the presence of certain types of plaque, and are relatively costly. This approach also has never been adopted or considered to be a viable approach to reducing the devastating impact that heart disease has on the general public.

There is therefore a need and it is an object of the present inventions to provide an accurate and non-invasive method and assembly which determines the likelihood of the existence of heart disease within an individual.

There is therefore a further need for an accurate non-invasive method and an assembly to predict the occurrence of heart disease within an individual in order to allow such early pro-active life-saving intervention to be accomplished on that individual.

### SUMMARY OF THE INVENTIONS

It is a first non-limiting object of the present inventions to provide a new and novel method and assembly to predict the occurrence of heart disease within an individual and which overcomes the drawbacks of prior methods, assemblies, and approaches.

It is a second non-limiting object of the present inventions to provide a new and novel method and assembly to predict the occurrence of heart disease within an individual by the use of both image and measurement data.

It is a third non-limiting object of the present inventions to provide a new and novel method and assembly to predict the occurrence of heart disease within an individual by the use of non-invasive echo images and real time measurement data and which may be accomplished in a non-invasive manner.

According to a first non-limiting aspect of the present inventions, an assembly to predict the occurrence of heart disease within an individual is provided. Particularly, the assembly includes a sensor portion which measures at least one bodily metric of the individual and which transmits the measurement; and a second portion which receives the transmitted measurement from the first portion and which utilizes the received transmitted measurement and at least one image of the heart of the individual to predict whether the individual has heart disease.

According to a second non-limiting aspect of the present invention, a method is provided for predicting the occurrence of heart disease within an individual. The method includes the steps of measuring at least the blood pressure of the individual; acquiring an image of the heart of the individual; and using both the image of the heart and the measured blood pressure to predict whether the individual has heart disease.

These and other aspects, features, and advantages of the present inventions will become apparent from a reading of the following detailed description of the preferred embodiment of the invention, including the subjoined claims, and by reference to the following drawings.

### BRIEF DESCRIPTION OF THE DRAWINGS

FIG. 1 is a block diagram if the method and assembly of the preferred embodiment of the invention.

FIG. 2 is an information table resident within the computer assembly shown in FIG. 1 and used by the computational software portion of the computer assembly to predict the occurrence of heart disease within an individual.

### DETAILED DESCRIPTION OF THE PREFERRED EMBODIMENT OF THE INVENTION

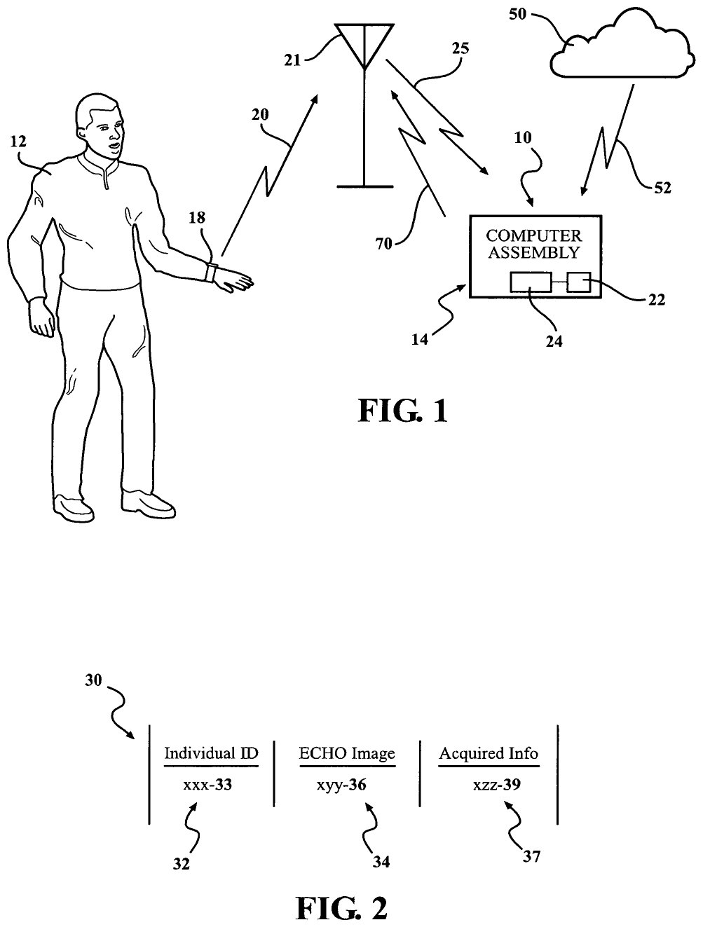

Referring now to FIG. 1, there is shown assembly 10 which is made in accordance with the teachings of the preferred embodiment of the inventions and which is used within the preferred method of the various inventions to predict the occurrence of heart diseases within an individual, such as individual 12. It should be apparent that individual 12 represents any individual who a prediction is to be made by assembly 10 of the occurrence of heart disease and that the present inventions are not limited to any particular individual or type of individual.

As shown, assembly 10 includes a computer assembly 14 which operates under stored program control and the operation of the stored program is more fully described later in this description.

The individual 12 is made to wear at least one device 18 which senses at least one bodily metric of the individual 12, such as by way of example and without limitation, the blood pressure and the heart rate of the individual 12. As used herein, the term “bodily metric” means anything which can be measured and which relates to or is associated with any function or operation of the body (or any constituent element or portion of the human body) of the individual 12 (e.g., blood pressure relates to and is associated with the function of blood flow within the body of the individual 12 and heart rate relates to and is associated with the beating of the heart of the individual 12). This sensed information 20 is then transmitted to the computer assembly 14. Such transmission may occur by the use of a cellular signal 20 which is communicated to one or more cellular towers 21, by radio frequency or by Bluetooth®, or by similar known transmission mechanisms. The device 18 may comprise a watch manufactured and sold by Apple® or any other similar known and commercially available health wearable devices. Generally, the device 18 may comprise anything which senses any bodily metric of the individual 12 and which allows communication of the sensed bodily metric to the computer assembly 14.

The computer assembly 14 includes a storage portion 22 and a processor portion 24 in communication with the storage portion 22. The processor portion 24 uses the data resident within the file storage portion 22 to predict the occurrence of heart disease within the individual 12. The code or operational set of instructions used by the processor portion 24 to perform its prognostication functions may also reside within the storage portion 22. The information 20 is received by the processor portion 24 by, for example, a cellular signal 25 from the one or more cellular towers 21, or from other types of known communication signals (e.g., radio frequency or Bluetooth®).

As shown best in FIG. 2, the file storage portion 22 includes a file 30 associated with individual 12 and the file 30 includes a first data entry 32 comprising a unique identification number or alpha-numeric identifier 33 of the individual 12; a second data entry 34 comprising the latest echo cardiogram image 36 of the individual 12; and a third data entry 36 comprising the latest bodily metric data 39 which was received by the processor 24. In one non-limiting example, the bodily metric data 39 comprises the blood pressure and/or heart rate data of the individual 12 which was received from the device 18. It should be appreciated that data 39 may be updated as new information 20 is generated from device 18 and entry 34 (data 36) may similarly be updated as new echo cardiograms are performed upon individual 12. As the data entries 34, 37 are updated, the processor portion 24 performs new predictions or prognostications. It should be appreciated that the creation of an echo cardiogram is both cost effective and non-invasive and the information 39 is also acquired in a cost effective and non-invasive manner. Thus, the computer assembly 14 includes both echocardiogram data 36 and bodily metric data 39 of the individual 12 and this data is accessible by the processor 24 which uses the data in accordance with the instructions found within the storage portion 22 to make a prognostication as to whether the individual 12 has a heart disease condition. These prognostications may be repeated over time as new echocardiogram and/or bodily metric data is received by the computer assembly 12 in the manner, for example, set forth above.

In a first non-limiting embodiment of the invention, processor 24 will prognosticate that heart disease is present in the individual 12 of the results of a transthoracic echocardiogram (e.g., comprising data 36 of entry 34) show a greater than 50% narrowing in any of the coronary arteries, and will also prognosticate the presence of heart disease in the individual 12 if there is between a 35 and 50% narrowing of any of the coronary arteries and the measured blood pressure (comprising data 39 of entry 37) has a systolic value which is equal to or greater than 140 or a diastolic value which is equal to or greater than 90. Otherwise, the processor 24 will prognosticate that heart disease is not present. This is only one non-limiting operational algorithm which may be used by the methodology and assembly of the present invention. The prognostication will be communicated to user or individual 12 by the generation of signal 70, by the processor 24, and the communication of the generated signal 70 to the individual 12 by the use of tower 21 or by the use of any other known communication methodology.

In one non-limiting embodiment of the inventions, the echo cardiogram images (e.g., they each may be of the transthoracic type) are processed by the CNN-SNet model (Convolutional Neural Network with SqueezeNet) (“The Model”) using the UC Irvine Machine Learning Repository dataset for training and validating the process occurring within the processor portion 24. The UC Irvine Machine Learning Repository dataset (“UCI Set”) is available, for example, at https://archive.ice.uci.edu/ml/datasets.ohp. The echo-cardiogram portion of The UCI Set is available at https://archive.ice.uci.edu/ml/datasets/Echocardiogram.

That is, the received echo cardiogram images are one input to The Model and The Model uses this input together with the echo-cardiogram portion of The UCI Set and the Model then provides a probability of heart disease based solely on the received echo-cardiogram image. However, the preferred embodiment of the invention adds to the accuracy of this result by use of the wearable device information which provides the previously described bodily metric information of the individual 12.

The acquired information 39 (comprising entry 37) received from the wearable device 18 is used within the known and commercially available SWM-MWO model (Support Vector Machine (SWM) with Modified Whale Optimization) (“The Second Model”) and this Second Model, using the wearable data also provides an independent prognostication of the probably occurrence of heart disease by using only the wearable information (e.g., blood pressure and heart rate).

According to the teachings of the most preferred embodiment of the invention, the processor 24 combines the probability outputs of The First and The Second Models to make a final determination as to whether the individual 12 actually has a heart disease condition. In one non-limiting embodiment, the processor portion 24 will prognosticate that the individual 12 has heart disease only if each of the Models provide a probability greater than or equal to 50% that heart disease is present within the individual 12. Otherwise, the processor portion 24 will prognosticate that heart disease is not present within the individual 12. Other prognostication thresholds may be utilized in other non-limiting embodiments of the inventions. In yet another non-limiting embodiment of the inventions, as earlier described, processor 24 will cause computer assembly 14 to generate a prognostication signal 70 to the user 12 (e.g., by use of the tower 21 or another communications medium) informing the user 12 of the results of the prognostication of the assembly 14.

In one non-limiting embodiment of the inventions, the prediction model, used by the processor portion 24, resides within the storage portion 22 and may be developed as follows. In the first phase of the prediction model development, sensors such as a blood pressure sensor, pulse oximeter sensor, and echocardiogram sensor/apparatus are placed upon the individual 12. The obtained medical data from each of these sensors is then transmitted to the processor 24 by Bluetooth® or another known technique and saved within the storage portion 22 as binary and comma-separated values (.csv) files. This data is also stored within a secure cloud storage environment 50 which is in communication with processor 24, by a known type of signal 52, such as radio frequency signal. The secure cloud storage environment 50 allows for a doctor or other medical professional to verify or validate the acquired data. The cloud stored data is then retrieved by the processor 24 and processed for medical data classification by using the known Hybrid model SVM-MWO algorithm, creating a “phase 1 module”.

In the second phase of the prediction model development, a customized echocardiogram heart disease dataset is collected and stored in the secure cloud storage environment 50. Such a dataset, for example and without limitation, may be obtained from public databases and classified according to the publicly available Hybrid CNN-Snet Algorithm for medical image classification, thereby creating a “phase 2 module”. Both the phase 1 and the phase 2 modules are then trained with publicly available datasets from the known UCI repository for evaluation.

After the foregoing has been completed, a testing phase is then accomplished. In this testing phase, each of the phase 1 and the phase 2 modules individually respectively perform classification on the acquired data 20 and on the acquired echocardiogram data. The separately classified data and images are combined and validated in order to predict whether the individual associated with the acquired data has heart disease, thereby obviating the need for a doctor to make this determination or prediction.

An example of the development of these phases is as follows:

Data was collected from a patient included the patient's name; age; gender; Echocardiogram values as follows: QRS-100 ms, QT/QTeBaz:398/370 ms, PR: 156 ms, P: 84 ms; RR/PP:1158/1153 ms, P?QRS/T:7/63/37 degrees. Axis: Normal QRS axis, and Intervals: PR, QRS, and QT intervals were normal; temperature of 98.3 F; blood pressure of 128/84; and pulse rate of 54 bpm, variable.

It was determined that the foregoing patient data represented a normal echocardiogram reading, normal temperature, normal heart rate, and a normal blood pressure. Normally the heart rhythm is of the sinus-type characterized, among other features, by the presence of P-complex before every QRS complex in a cycle and simultaneously the presence of QRS after every P complex. The heart impulse rate is characterized by heart agitation in average in the frequency of 60-100 beats per minute (bpm) for adults, although it might be normal, especially for athletes, to have a resting bpm as low as 40. Deviation of the rate beyond this range can be treated as an abnormal case. However, the limit frequencies should be taken individually. For the time duration of PR, QRS, and QT parts in the cardiac cycle, a reasonable rule is to consider the interval QT being less than half of the distance between two successive QRT complexes. That is, QT should be less than one-half of the RR interval. The P complex (P wave) is normally 0.04-0.11 s in duration. Its deviation from the normal wave shape or its disappearing means a pathological case. The normal duration of a ST segment is 0.02-0.12 s. Any drop in the duration of the ST segment predicted is ischemic, while the shift above the cycle-axis predicted as heart attach or abnormal.

The process, in the most preferred embodiment of the invention works in two individual phases. While the results of these two phases were combined to predict heart disease. Mostly by the results of phase 1, the user will know the impact of the disease by observing the echocardiogram, heart rate, and the blood pressure. For the detailed diagnosis and the guidance of the doctor, the user must undergo an echocardiogram imaging diagnosis. The doctor will observe the results of the phase 1 and guide the patient for further diagnosis in phase-2. Both the doctor and the user can monitor the results remotely. In case of an emergency, the user must visit a hospital typically recommended by a doctor.

The process/assembly of the invention handles two different types of medical data (signal and images). Both of these types of data were and are processed separately, and the results are combined to analyze heart disease. The classification results are combined to predict heart disease and whether the disease is present or not. The main significance of this system 10 is that most of the existing systems are made only to operate on either medical image or medical signal classification. But this system 10 is an integration of classifying both the sensor data and the medical image data for the prediction of heart disease. In normal class (health) classification, the proposed SVM-MWOA method achieved a 98.45% accuracy, a 96.83% precision, a 94.20% recall, and a 93.05% f-score on the Cleveland dataset, and in the abnormal class (unhealthy) classification, the proposed SVM-MWOA method achieved a 98.45% accuracy, a 96.83% precision, a 91.45% recall, and a 92.37% f-score using the medical and signal data collected by the foregoing described sensors. The Cleveland dataset is publicly available at https://archive.ics.uci.edu/ml/satasets/heart-disease. The proposed CNN with SqueezeNet classifier obtained the performance of a 99.09% accuracy, a 94.76% precision, a 98.95% recall, and a 98.60% F-score in classifying echocardiogram images. In contrast to the desirable performance of the SqueezNet Classifier embodiment of the present invention, the AlexNet Algorithm provides only a 96.08 accuracy; a 89.45 precision; a 93.43 recall; and a 90.81 F-Score and the VGG-16 algorithm provides only a 95.36 accuracy; a 88.43 precision; 95.20 recall; and a 92.45 F-score. The ResNet-101 algorithm provides only a 96.51 accuracy; a 89.64 precision; a 94.01 recall; and a 91.28 F-score. The Inception-v3 algorithm provides only a 97.80 accuracy; a 88.10 precision; a 96.48 recall; and a 95.13 F-score. The Xception algorithm provides only a 97.54 accuracy; a 89.78 precision; a 96.91 recall; and a 93.47 F-score. The DenseNet201 algorithm only provides a 98.48 accuracy; a 90.21 precision; a 98.54 recall; and a 94.24 F-score. Thus, according to Applicant's testing and findings the present and previously explained CNN-SqueezeNet embodiment achieves a far superior result over known competing algorithmic embodiments and this superior result was not expected at the outset.

It is to be understood that the various inventions are not limited to the exact construction or method which has been illustrated and discussed above, but that various changes and modifications may be made without departing from the spirit and the scope of the inventions as set forth, for example, in the following claims.

What is claimed is:

1) A assembly to predict the occurrence of heart disease within an individual comprising a sensor portion which measures at least one bodily metric of an individual and which transmits the measurement; a second portion which receives said transmitted measurements from said first portion and which utilizes said received transmitted measurements and at least one image of the heart of said individual to predict whether said individual has heart disease.

2) The assembly of claim 1 wherein said at least one bodily metric comprises a blood pressure of said individual.

3) The assembly of claim 2 wherein said at least one bodily metric comprises a heart rate of said individual in addition to said blood pressure.

4) The assembly of claim 3 wherein said image of said heart comprises a transthoracic image.

5) The assembly of claim 4 wherein said assembly further transmits a prediction signal to said individual.

6) A method for predicting the occurrence of heart disease within an individual comprising the steps of measuring at least one bodily metric of said individual; acquiring an image of the heart of said individual; and using both said image of said heart and said at least one bodily metric to predict whether said individual has heart disease.

7) A method for predicting whether an individual has heart disease comprising the steps of acquiring a blood pressure of said individual; acquiring an echocardiogram of the heart of said individual; and using said blood pressure to predict whether said individual has heart disease.
